(19) United States
(12) Patent Application Publication
(10) Pub. No.: US 2026/0335622 A1
(43) Pub. Date: Oct. 29, 2026

(54) HEMOSTASIS CAP FOR INTRODUCER SHEATH

(71) Applicant: BOSTON SCIENTIFIC SCIMED, INC., MAPLE GROVE, MN (US)

(72) Inventors: Todd Rowe, Excelsior, MN (US); Allison Michelle Marsh, Minneapolis, MN (US); Steven Thomas Albers, Maple Grove, MN (US)

(73) Assignee: BOSTON SCIENTIFIC SCIMED, INC., MAPLE GROVE, MN (US)

(21) Appl. No.: 19/660,811

(22) Filed: Apr. 28, 2026

Related U.S. Application Data
(60) Provisional application No. 63/795,842, filed on Apr. 28, 2025.

(51) Int. Cl. A61M 39/22; A61M 5/32

(52) U.S. Cl. CPC A61M 39/22; A61M 5/3293; A61M 2207/00

(57) ABSTRACT

A vascular access system includes an elongate shaft having a lumen and a hub body coupled to the elongate shaft. The hub body has a central port and a side port. The system further includes at least one valve disposed within the hub body and a cap disposed on the hub body. The cap includes a top surface with a central opening and at least one cutout extending radially outward from the central opening.

### CROSS-REFERENCE TO RELATED APPLICATIONS

This application claims the benefit of priority of U.S. Provisional Application No. 63/795,842 filed Apr. 28, 2025, the entire disclosure of which is hereby incorporated by reference.

### TECHNICAL FIELD

The disclosure is directed to a single access introducer sheath. More particularly, the disclosure is directed to an introducer sheath with a cap with a central and offset opening.

### BACKGROUND

A wide variety of intracorporeal medical devices have been developed for medical use, and more specifically for intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing methods, and use alternatives for medical devices.

In an example, a vascular access system may include an elongate shaft having a lumen, a hub body coupled to the elongate shaft where the hub body might have a central port and a side port, a cap disposed on the hub body where the cap may include a top surface with a central opening and at least one cutout extending radially outward from the central opening, and at least one valve that might be disposed within the cap.

Alternatively or additionally to any of the examples above, in another example, the central opening may be coaxial with the lumen of the elongate shaft.

Alternatively or additionally to any of the examples above, in another example, the at least one cutout might be aligned with the side port.

Alternatively or additionally to any of the examples above, in another example, the at least one valve may be disposed directly under the cap.

Alternatively or additionally to any of the examples above, in another example, the at least one valve might include three valves.

Alternatively or additionally to any of the examples above, in another example, the central opening may have a diameter of between 5 mm and 20 mm.

Alternatively or additionally to any of the examples above, in another example, the at least one cutout might be a semicircular extension of the central opening.

Alternatively or additionally to any of the examples above, in another example, the at least one cutout may include two cutouts positioned opposite one another across the central opening.

Alternatively or additionally to any of the examples above, in another example, the at least one cutout might include four spaced apart cutouts extending radially outward from the central opening.

Alternatively or additionally to any of the examples above, in another example, the central opening and the at least one cutout may each have a sloped edge.

In an example, a vascular access system might include an elongate shaft having a lumen, a hub body coupled to the elongate shaft where the hub body may have a central port, a cap disposed on a proximal end of the hub body where the cap might have a top surface with a central opening and at least one cutout extending radially outward from the central opening, and a plurality of valves that may be disposed within the cap.

Alternatively or additionally to any of the examples above, in another example, the central opening might be coaxial with the lumen of the elongate shaft.

Alternatively or additionally to any of the examples above, in another example, the hub body may include a side port and the at least one cutout might be aligned with the side port.

Alternatively or additionally to any of the examples above, in another example, the plurality of valves may include three valves, each having at least one slit, where each of the plurality of valves might be stacked one on top of another with the at least one slit on each valve offset from the slits in adjacent valves.

Alternatively or additionally to any of the examples above, in another example, the central opening may have a diameter of between 5 mm and 20 mm.

Alternatively or additionally to any of the examples above, in another example, the at least one cutout might be a semicircular extension of the central opening.

Alternatively or additionally to any of the examples above, in another example, the at least one cutout may include two cutouts positioned opposite one another across the central opening.

Alternatively or additionally to any of the examples above, in another example, the at least one cutout might include four spaced apart cutouts extending radially outward from the central opening.

Alternatively or additionally to any of the examples above, in another example, the central opening and the at least one cutout may each have a sloped edge.

In an example, a vascular access system might include an elongate shaft having a lumen, a hub body coupled to the elongate shaft where the hub body may have a central port and a side port, a cap disposed on the hub body where the cap might include a body having a top surface with a central opening and at least one cutout extending radially outward from the central opening where the central opening may be coaxial with the lumen of the elongate shaft, and at least one valve that might be disposed within the cap.

The above summary of some example embodiments is not intended to describe each disclosed embodiment or every implementation of the invention.

### BRIEF DESCRIPTION OF THE DRAWINGS

The invention may be more completely understood in consideration of the following detailed description of various embodiments in connection with the accompanying drawings, in which:

FIG. 1 is a side cross-sectional view of an example vascular access system with a cap;

FIG. 2 is an exploded view of the valves in FIG. 1;

FIG. 3A is a perspective view of the cap in FIG. 1;

FIG. 3B is a top view of the cap in FIG. 3A;

FIG. 3C is a top view of another example cap;

FIG. 4A is a cross-sectional view of the vascular access system of FIG. 1 with a catheter shaft and needle inserted into the cap;

FIG. 4B is a perspective view of the system of FIG. 4A;

FIG. 5A is a perspective view of another example cap; and

FIG. 5B is a top view of the cap in FIG. 5A.

While the invention is amenable to various modifications and alternative forms, specifics thereof have been shown by way of example in the drawings and will be described in detail. It should be understood, however, that the intention is not to limit aspects of the invention to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions, ranges and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the invention. The illustrative embodiments depicted are intended only as exemplary. Selected features of any illustrative embodiment may be incorporated into an additional embodiment unless clearly stated to the contrary.

Transcatheter aortic valve replacement (TAVR) access sheaths may have a single hemostasis valve to accommodate a delivery system. The TAVR procedure generally requires use of a pigtail catheter that typically has a separate access sheath. In some cases, it may be desired to perform a TAVR and reduce the number of vessel access sites required by using a single access sheath for both the aortic valve delivery system and guide catheter used in the procedure. This procedure may be desired for patients that have severe tortuosity in one of their femoral arteries, limiting access to a single side. Conventional hemostasis valves may have a central crosscut to minimize leakage around an inserted device. However, for a single access procedure, the valve would also be punctured with a needle to insert a guide catheter. In current systems, there is not much space adjacent to the center crosscut to perform this puncture. A cutout in the cap that secures the hemostasis valve to the hub provides more space for this needle puncture.

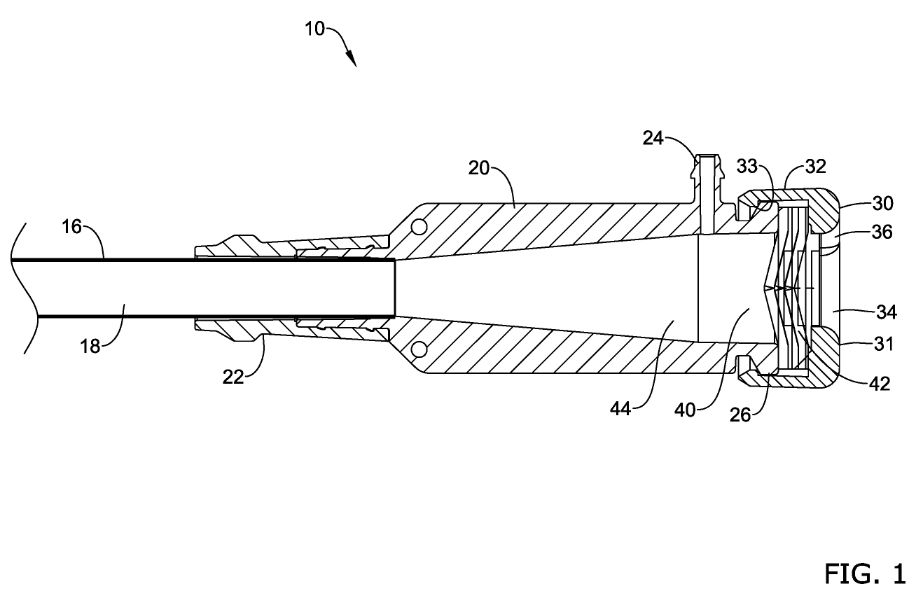

FIG. 1 is a cross-sectional view of an illustrative vascular access system 10. The vascular access system 10 may be used to gain vessel access during an interventional procedure, such as a transcatheter aortic valve replacement (TAVR) procedure. While the access system 10 is described in terms of vascular access, the access system 10 may be used to access other parts of the body. The access system 10 may include an elongate shaft 16 defining a lumen 18, a hub body 20 coupled to the elongate shaft 16, at least one hemostasis valve 42, and a cap 30. The elongate shaft 16 may have a size (outside diameter or profile) ranging from 4 French (F) to 9F, and a length ranging from 10 centimeters (cm) (3.94 inch) to 25 cm (9.84 inches). However, the outer diameter can be smaller than 4F or greater than 9F. It is further contemplated that the length of the elongate shaft 16 may be shorter than 10 cm or longer than 25 cm.

In some embodiments, the access system 10 may include a strain relief portion 22 coupled to or formed as a unitary structure with the hub body 20 and extending distally therefrom. The hub body 20 may include a central port 40 defined by a cavity 44, and a side port 24 for connection to a flush or injection tube subassembly (not explicitly shown). The valve(s) 42 may be positioned in the cavity 44 of the hub body 20, adjacent the central port 40, or they may be disposed within the cap 30. In some embodiments, one of the valves 42 may be positioned directly under the cap 30, as shown in FIG. 1.

The cap 30 may be removably coupled to the hub body 20 and may aid in securing the one or more hemostasis valve(s) 42. The hemostasis valve(s) 42 may reduce or prevent blood loss when the elongate shaft 16 is inserted into a vessel while allowing for the passage of other devices (e.g., dilators, guide catheters, etc.) through the cap 30, valve(s) 42 and elongate shaft 16. Materials for the valve(s) may include, but are not limited to, a liquid silicone rubber (LSR) elastomer or enhanced tear resistant (ETR) silicone elastomers. The hemostasis valve 42 may include one or more interlocking features configured to engage mating features on the hub body 20 to retain the hemostasis valve in a desired position and/or orientation within the hub body 20 (e.g., at a fixed longitudinal and/or axial position within the cavity 44), such as those described in U.S. Pat. No. 11,779,743, entitled, “Hemostasis Valve Design for Introducer Sheath” to Agrawal et al., which is incorporated by reference herein.

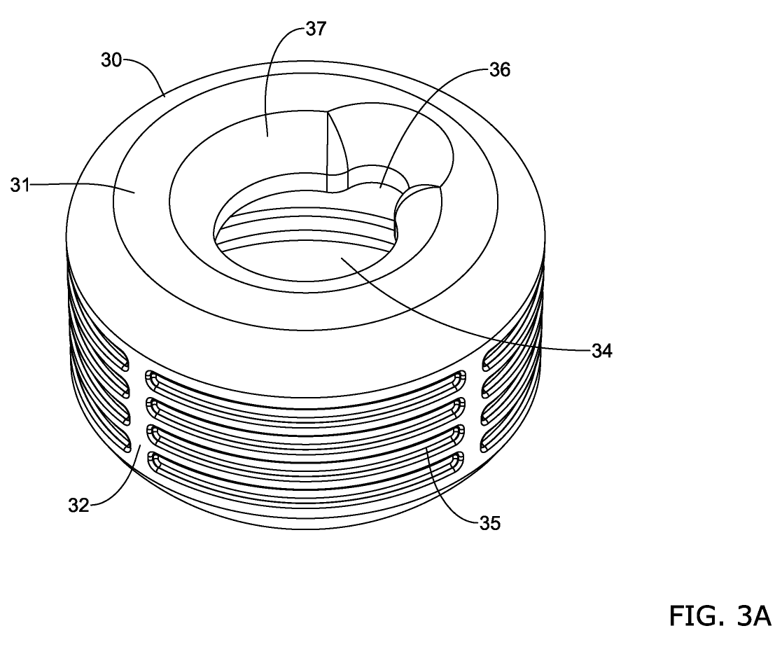

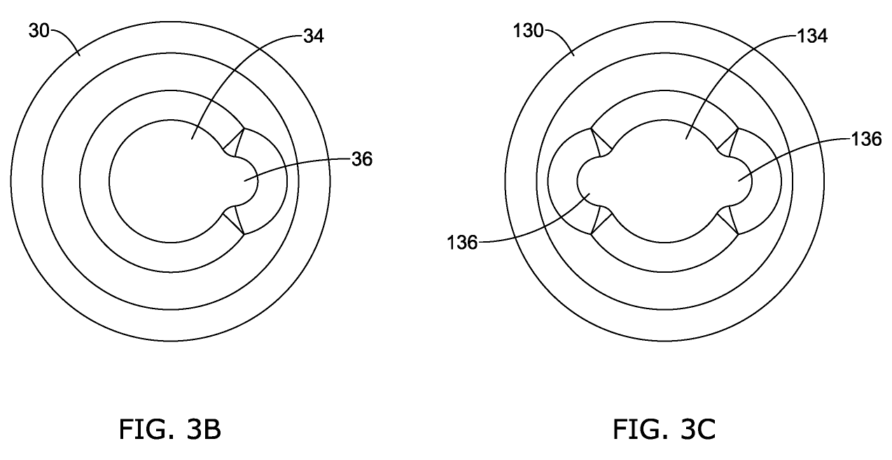

The cap 30 may have a top surface 31 and a circumferential sidewall 32 extending distally therefrom. The sidewall 32 may include a plurality of grooves 35 (shown and described in greater detail in FIG. 3A) to aid in griping the cap 30 when attaching it to the hub body 20. The cap 30 may be coupled to the proximal end of the hub body 20 by a flange 33 extending radially inward adjacent the distal end of the circumferential sidewall 32. The flange 33 may be configured to engage a radially outward protrusion 26 at the proximal end of the hub body 20 in a friction or snap fit. The cap 30 may include a central opening 34 extending completely through the top surface 31. The central opening 34 may be coaxial with the lumen 18 of the elongate shaft 16. The cap 30 may further include at least one cutout 36 extending radially outward from the central opening 34. In some embodiments, the cutout 36 may be aligned with the side port 24, as shown in FIG. 1. In other embodiments, the cutout 36 may be disposed at other positions, such as opposite the side port 24 or at two to three o’clock position relative to the side port.

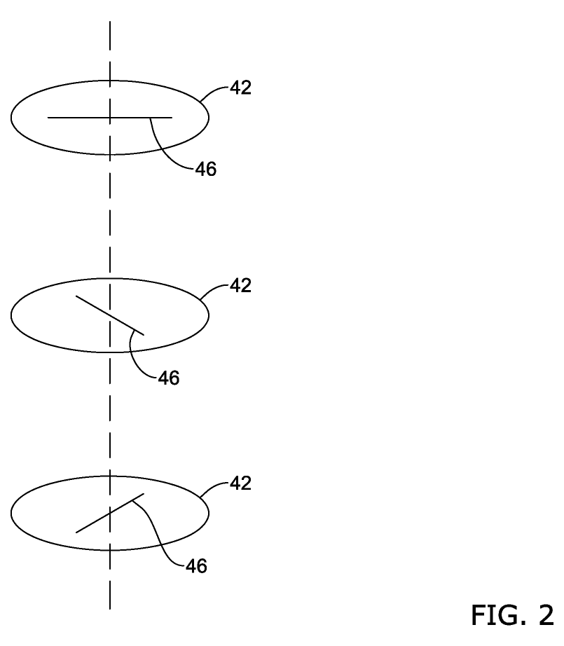

As shown in FIG. 1, three valves 42 may be stacked within the cavity 44. The three valves 42 may each have at least one slit 46, and each of the valves is stacked one on top of another with the at least one slit 46 on each valve offset from the slits in the other valves. In some embodiments, the slit 46 may extend fully through the thickness of the valve 42. In other embodiments, the slit 46 may extend partially through the thickness of the valve 42. For example, each valve 42 may have a first slit 46 on a first side that extends only partially through the thickness, and a second slit 46 perpendicular to the first slit, with the second slit extending partially through the thickness on the second, opposite side, such that the two slits meet to create a single point of opening extending all the way through the valve 42 in its center. In some embodiments, a plurality of valves 42 may be stacked with the slits 46 on the first side of each valve offset from one another, as shown in FIG. 2 which is an exploded view of the valves in FIG. 1.

Further details of the cap 30 are illustrated in FIGS. 3A and 3B. FIG. 3A is a perspective view of the cap 30 in FIGS. 1 and 3B is a top view of the cap 30 in FIG. 3A. The central opening 34 may be circular and may have a diameter of between 5 millimeters (mm) (0.197 inch) and 20 mm (0.787 inch). The cutout 36 may be a semicircular extension of the central opening 34, as shown in FIGS. 3A and 3B. In other embodiments, the cutout 36 may be oval, oblong, stadium shaped, or have any other curved shape. The central opening 34 and the cutout 36 may each have a sloped edge 37 that slopes inward and downward from the top surface 31. The sloped edges 37 may help guide insertion of a catheter shaft or needle into the central opening 34 and/or cutout 36, respectively. FIG. 3C is a top view of another example cap 130. In this embodiment, the cap 130 has a central opening 134 and two cutouts 136 positioned opposite one another across the central opening 134. This embodiment provides an additional location for insertion of a needle or auxiliary catheter shaft.

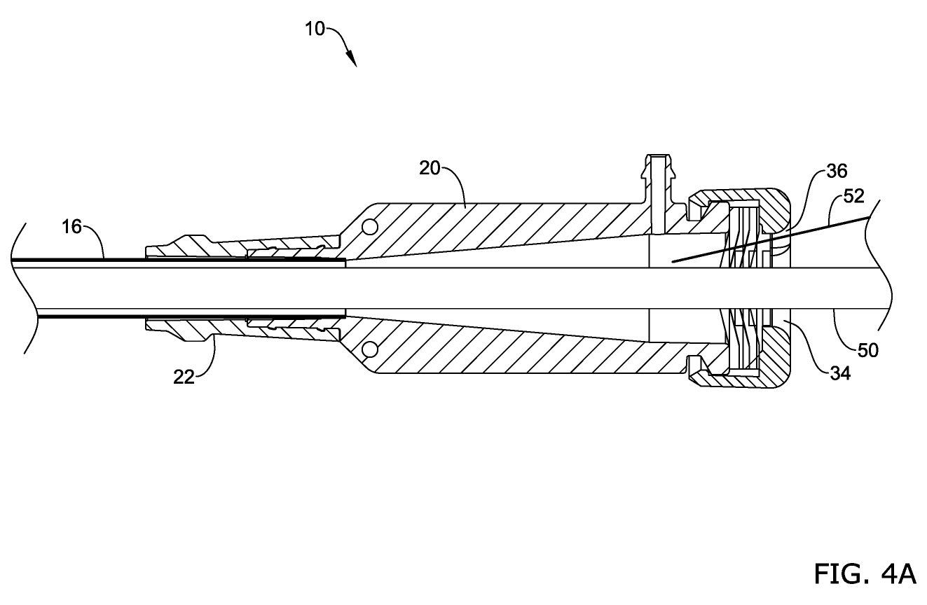

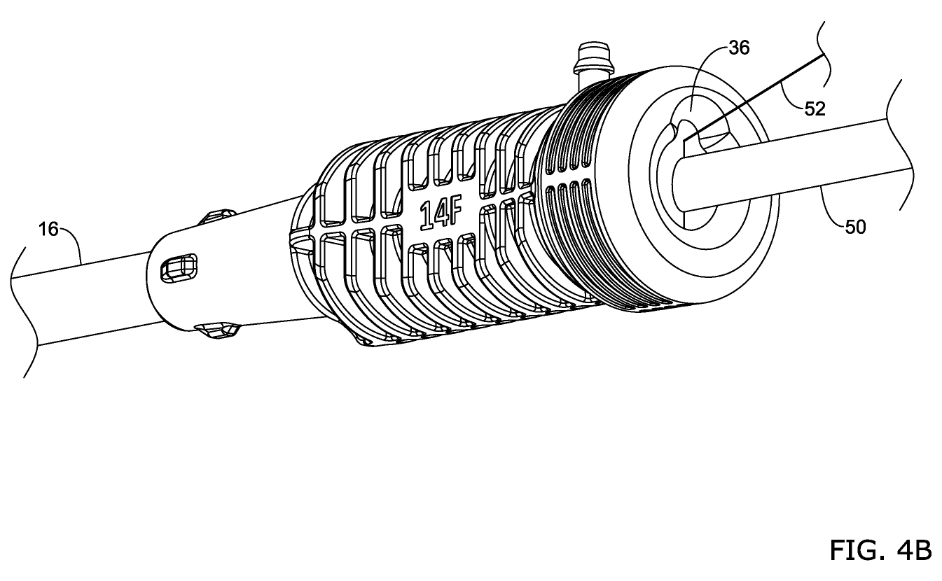

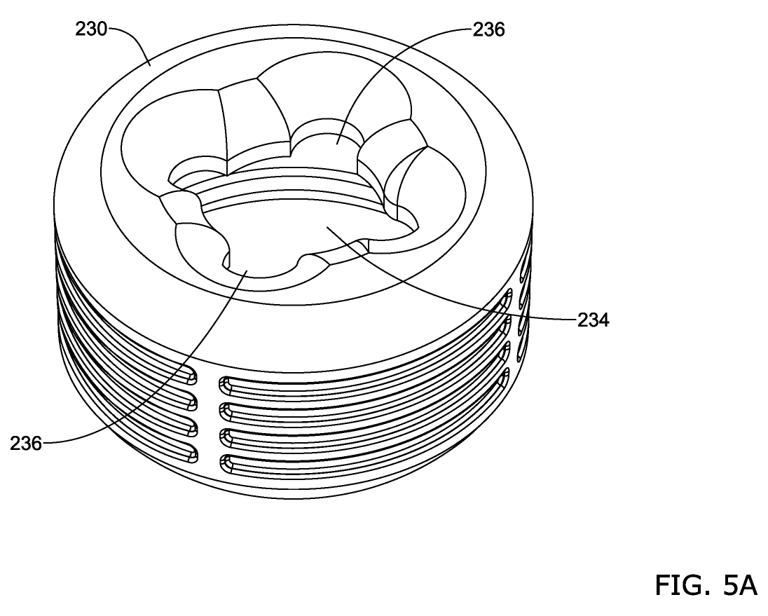

The cutout 36 in the cap 30 may enable single access procedures to be performed by providing space for physicians to perform a needle puncture of the hemostasis valve 42 for insertion of a guide catheter through the cap 30 next to a TAVR delivery system that is passed through the central opening 34 of the cap 30 and hemostasis valve 42 as normal. FIGS. 4A and 4B illustrate the access system 10 of FIG. 1 with a catheter shaft 50, such as that used in a TAVR delivery system, inserted through the central opening 34 in the cap and through the valves. Also shown is a needle 52 inserted through the cutout 36 and valves. The cutout 36 provides additional space for the needle 52 to be inserted which facilitates additional guidewires and/or catheter shafts to be inserted through the hub body 20 and elongate shaft 16 while the catheter shaft 50 remains in place.

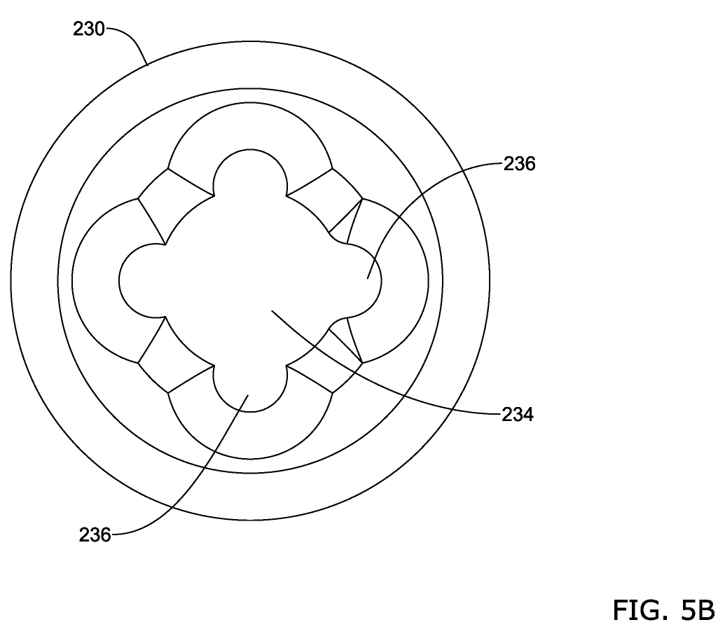

FIG. 5A is a perspective view of another example cap 230 and FIG. 5B is a top view of the cap 230 in FIG. 5A. The cap 230 has four cutouts 236 extending radially outward from and spaced apart evenly around a central opening 234. In other embodiments, three cutouts may be provided, creating a triangular opening.

The materials that can be used for the various components of the vascular access system 10 (and/or other systems disclosed herein) and the various elements thereof disclosed herein may include those commonly associated with medical devices. For simplicity purposes, the following discussion refers to the access system 10. However, this is not intended to limit the devices and methods described herein, as the discussion may be applied to other elements, members, components, or devices disclosed herein, such as, but not limited to, the elongate shaft 16, hub body 20, cap 30 and the hemostasis valves 42, and/or elements or components thereof.

In some embodiments, the access system 10, and/or components thereof, may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated "linear elastic" or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial "superelastic plateau" or "flag region" in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear than the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about –60 degrees Celsius (ºC) to about 120 ºC in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, portions or all of access system 10, and/or components thereof, may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the access system 10 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the access system 10 to achieve the same result.

In some embodiments, an exterior surface of the access system 10 (including, for example, an exterior surface of the delivery system) may be sandblasted, bead blasted, sodium bicarbonate-blasted, electropolished, etc. In these as well as in some other embodiments, a coating, for example a lubricious, a hydrophilic, a protective, or other type of coating may be applied over portions or all of the outer sheath, or in embodiments without an outer sheath over portions of the delivery system, or other portions of the access system 10. Hydrophobic coatings such as fluoropolymers provide a dry lubricity which improves device handling and device exchanges. Lubricious coatings improve steerability and improve lesion crossing capability. Suitable lubricious polymers are well known in the art and may include silicone and the like, hydrophilic polymers such as high-density polyethylene (HDPE), polytetrafluoroethylene (PTFE), polyarylene oxides, polyvinylpyrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility.

The coating and/or sheath may be formed, for example, by coating, extrusion, co-extrusion, interrupted layer co-extrusion (ILC), or fusing several segments end-to-end. The layer may have a uniform stiffness or a gradual reduction in stiffness from the proximal end to the distal end thereof. The gradual reduction in stiffness may be continuous as by ILC or may be stepped as by fusing together separate extruded tubular segments. The outer layer may be impregnated with a radiopaque filler material to facilitate radiographic visualization. Those skilled in the art will recognize that these materials can vary widely without deviating from the scope of the present invention.

It should be understood that this disclosure is, in many respects, only illustrative. Changes may be made in details, particularly in matters of shape, size, and arrangement of steps without exceeding the scope of the invention. This may include, to the extent that it is appropriate, the use of any of the features of one example embodiment being used in other embodiments. The invention's scope is, of course, defined in the language in which the appended claims are expressed.

What is claimed is:

1. A vascular access system, comprising:
an elongate shaft having a lumen;
a hub body coupled to the elongate shaft, the hub body having a central port and a side port;
a cap disposed on the hub body, the cap including a top surface with a central opening and at least one cutout extending radially outward from the central opening; and
at least one valve disposed within the cap.

2. The vascular access system of claim 1, wherein the central opening is coaxial with the lumen of the elongate shaft.

3. The vascular access system of claim 1, wherein the at least one cutout is aligned with the side port.

4. The vascular access system of claim 1, wherein the at least one valve is disposed directly under the cap.

5. The vascular access system of claim 4, wherein the at least one valve includes three valves.

6. The vascular access system of claim 1, wherein the central opening has a diameter of between 5 mm and 20 mm.

7. The vascular access system of claim 1, wherein the at least one cutout is a semicircular extension of the central opening.

8. The vascular access system of claim 1, wherein the at least one cutout includes two cutouts positioned opposite one another across the central opening.

9. The vascular access system of claim 1, wherein the at least one cutout includes four spaced apart cutouts extending radially outward from the central opening.

10. The vascular access system of claim 1, wherein the central opening and the at least one cutout each have a sloped edge.

11. A vascular access system, comprising:
an elongate shaft having a lumen;
a hub body coupled to the elongate shaft, the hub body having a central port;
a cap disposed on a proximal end of the hub body, the cap having a top surface with a central opening and at least one cutout extending radially outward from the central opening; and
a plurality of valves disposed within the cap.

12. The vascular access system of claim 11, wherein the central opening is coaxial with the lumen of the elongate shaft.

13. The vascular access system of claim 11, wherein the hub body includes a side port and the at least one cutout is aligned with the side port.

14. The vascular access system of claim 11, wherein the plurality of valves includes three valves, each having at least one slit, wherein each of the plurality of valves is stacked one on top of another with the at least one slit on each valve offset from the slits in adjacent valves.

15. The vascular access system of claim 11, wherein the central opening has a diameter of between 5 mm and 20 mm.

16. The vascular access system of claim 11, wherein the at least one cutout is a semicircular extension of the central opening.

17. The vascular access system of claim 11, wherein the at least one cutout includes two cutouts positioned opposite one another across the central opening.

18. The vascular access system of claim 11, wherein the at least one cutout includes four spaced apart cutouts extending radially outward from the central opening.

19. The vascular access system of claim 11, wherein the central opening and the at least one cutout each have a sloped edge.

20. A vascular access system, comprising:
an elongate shaft having a lumen;
a hub body coupled to the elongate shaft, the hub body having a central port and a side port;
a cap disposed on the hub body, the cap including a body having a top surface with a central opening and at least one cutout extending radially outward from the central opening, the central opening coaxial with the lumen of the elongate shaft; and
at least one valve disposed within the cap.
